(19) United States
(12) Patent Application Publication
(10) Pub. No.: US 2026/0090964 A1
(43) Pub. Date: Apr. 2, 2026

(54) COMPOSITION COMPRISING BIOSURFACTANT AND SALICYLIC ACID

(71) Applicant: L'OREAL, Paris (FR)

(72) Inventors: Christopher PANG, New York, NY (US); Changlong CHEN, Edison, NJ (US); Siliu TAN, Westfield, NJ (US); Mariko HASEBE, New York, NY (US)

(73) Assignee: L'OREAL, Paris (FR)

(21) Appl. No.: 18/901,492

(22) Filed: Sep. 30, 2024

Related U.S. Application Data
(63) Continuation of application No. 18/591,178, filed on Feb. 29, 2024.
(63) Continuation of application No. 18/591,930, filed on Feb. 29, 2024.

(30) Foreign Application Priority Data
Apr. 30, 2024 (FR) 2404520
Jun. 10, 2024 (FR) 2406067

(51) Int. Cl. A61K 8/365; A61K 8/02; A61K 8/60; A61Q 19/10

(52) U.S. Cl. CPC A61K 8/365; A61K 8/0212; A61K 8/602; A61Q 19/10

(57) ABSTRACT

The present invention relates to compositions comprising (a) at least one glycolipid; (b) at least one salicylic acid or its derivatives represented by formula (I); (c) at least one cationic polymer may be a polysaccharide derived from β-linked D-glucosamine and N-acetyl-D-glucosamine; (d) at least one clay; and (e) cosmetically acceptable solvents comprising water, glycerin. The compositions according to the present invention can provide an effect of treating, hydrating, and/or cleansing acne-prone skin.

### FIELD OF THE INVENTION

The present invention relates to compositions comprising at least one biosurfactant and at least one salicylic acid or its derivatives, preferably a cosmetic composition comprising the same.

### BACKGROUND ART

Anti-acne cleansing compositions often have the undesired effect of drying out the skin. Therefore, there is a need to develop an effective anti-acne composition which can provide sensorial and aesthetic benefits to acne prone-skin and also provide adequate hydration to the skin.

### DESCRIPTION

### Cross-Reference to Related Applications

This application claims benefit of U.S. Non-Provisional application Ser. No. 18/591,930, filed Feb. 29, 2024. This application also claims benefit of U.S. Non-Provisional application Ser. No. 18/591,178, filed Feb. 29, 2024, French Application No. FR2404520 filed Apr. 30, 2024 and French Application No. FR2406067 filed Jun. 10, 2024, all of which are incorporated herein by reference in their entirety.

### DISCLOSURE OF INVENTION

An objective of the present invention is to provide a composition for treating, hydrating and/or cleansing acne-prone skin.

The above objective of the present invention can be achieved by a composition comprising:
(a) at least one glycolipid;
(b) at least one salicylic acid or its derivatives of formula (I):

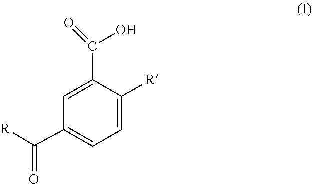

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

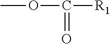

in which R₁ denotes a linear or branched, saturated or unsaturated aliphatic chain containing 1 to 18 carbon atoms; and also salts thereof derived from an inorganic or organic base;
(c) at least one cationic polymer selected from a polysaccharide derived from β-linked D-glucosamine or N-acetyl-D-glucosamine;
(d) at least one clay
(e) cosmetically acceptable solvents comprising water, and comprising glycerin present from about 2% to about 40% by weight, relative to the total weight of the composition; and wherein the pH of the composition is from about 3.5 to about 6.5.

The above objective of the present invention can also be achieved by a composition comprising:
(a) at least one rhamnolipid present from 2% by weight to 5% by weight, relative to the total weight of the composition;
(b) at least one salicylic acid or its derivatives present from about 1% by weight to about 3% by weight, relative to the total weight of the composition;
(c) at least one cationic polymer comprising chitosan;
(d) at least one clay comprising kaolin;
(e) cosmetically acceptable solvents comprising water, and comprising glycerin present from about 10% to about 30% by weight, relative to the total weight of the composition;
wherein the pH of the composition is from about 3.5 to about 6.5,
wherein the composition is suitable for 3-in-1 paste formulas for acne-prone skin as a skin cleanser, as a spot treatment, and as a mask.

The glycolipid is selected from the group consisting of rhamnolipids, sophorolipids, glucolipids, trehalolipids, cellobiose lipids and mixtures thereof. The glycolipid is preferably rhamnolipids.

The at least one salicylic acid or its derivatives is preferably salicylic acid.

The salicylic acid derivatives are represented by formula (I) in which the radical R denotes a linear, branched or cyclic, saturated aliphatic chain containing from 2 to 22 carbon atoms.

The salicylic acid derivatives are represented by formula (I) in which the R radical denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms.

The salicylic acid derivatives are represented by formula (I) in which R′ denotes a hydroxyl group.

The cationic polymer is a polysaccharide derived from β-linked D-glucosamine and N-acetyl-D-glucosamine, for example, chitosan.

The at least one clay, can be, for example, kaolin.

The cosmetically acceptable solvents comprise water, and comprise glycerin present from about 2% to about 40% by weight, relative to the total weight of the composition; or

The cosmetically acceptable solvents comprise water, and comprise glycerin present from about 10% to about 30% by weight, relative to the total weight of the composition.

The compositions according to the present invention may further comprise at least one surfactant comprising sodium cocoyl isethionate present at about 10% by weight, relative to the total weight of the composition.

The compositions according to the present invention may also further comprise niacinamide present at about 2% by weight, relative to the total weight of the composition. The pH of the composition is from about 3.5 to about 6.5.

The amount of the glycolipid(s), including rhamnolipids, may be 0.1% to 10% by weight, preferably 1% to 7%, by weight, more preferably 2% to 5% by weight, and in particular 3% by weight, relative to the total weight of the composition.

The amount of the salicylic acid or its derivatives may be from 0.1% to 5% by weight, preferably from 1% to 3% by weight, most preferably 2% by weight, relative to the total weight of the composition.

The amount of the clay may be from 5% to 25% by weight, preferably from 10% to 20% % by weight, most preferably 15% by weight, relative to the total weight of the composition.

The amount of the glycerin may be from 1% to 45% by weight, preferably from 2% to 40% % by weight, most preferably 10% to 30% by weight, relative to the total weight of the composition.

The pH of the compositions according to the present invention is from about 3.5 to about 6.5, preferably from about 4 to 6, most preferably from about 4.5 to 5.5, and particularly preferred pH is 5.5 or 5.0.

The compositions of the present invention preferably have a paste texture, particularly a unique wasabi-like paste texture. The wasabi-like paste may be used as a 3-in-1 composition for acne-prone skin, which includes a daily skin clearing cleanser to be mixed with water, an overnight drying spot treatment in its original form, and/or a mask when layered on damped face.

The compositions may be a cosmetic composition for treating, hydrating, and/or cleansing acne-prone skin.

The present invention also relates to method for treating, hydrating, and/or cleansing acne-prone skin, comprising:
applying onto the skin in need the compositions according to the present invention.

The present invention also relates to a use of a combination of the glycolipid(s), the salicylic acid or its derivative, the cationic polymer, the clay and the solvents comprising water and glycerine for treating, hydrating, and/or cleansing acne-prone skin.

### BEST MODE FOR CARRYING OUT THE INVENTION

After diligent research, the inventors have surprisingly discovered that compositions comprising at least one glycolipid, at least one salicylic acid or its derivatives having the specific structure of formula (I), at least one cationic polymer, at least one clay, and solvents comprising water and glycerine can treat, hydrate, and/or cleanse acne-prone skin, thus completing the present invention.

In one embodiment, the composition according to the present invention comprises:
(a) at least one glycolipid;
(b) at least one salicylic acid or its derivatives of formula (I):

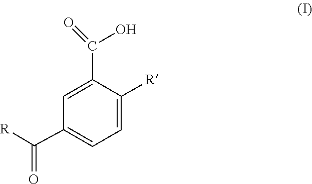

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

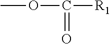

in which R₁ denotes a linear or branched, saturated or unsaturated aliphatic chain containing 1 to 18 carbon atoms;
and also salts thereof derived from an inorganic or organic base;
(c) at least one cationic polymer selected from a polysaccharide derived from β-linked D-glucosamine or N-acetyl-D-glucosamine;
(d) at least one clay;
(e) cosmetically acceptable solvents comprising water, and comprising glycerin present from about 2% to about 40% relative to the total weight of the composition; and
wherein the pH of the compositions is from about 3.5 to about 6.5.

In another embodiment, the composition according to the present invention comprises:
(a) at least one rhamnolipid present from 2% by weight to 5% by weight, relative to the total weight of the composition;
(b) at least one salicylic acid or its derivatives present from about 1% by weight to about 3% by weight, relative to the total weight of the composition;
(c) at least one cationic polymer comprising chitosan;
(d) at least one clay comprising kaolin;
(e) cosmetically acceptable solvents comprising water, and comprising glycerin present from about 10% to about 30% by weight, relative to the total weight of the composition;
wherein the pH of the composition is from about 3.5 to about 6.5,
wherein the composition is suitable for 3-in-1 paste formulas for acne-prone skin as a skin cleanser, as a spot treatment, and as a mask.

Hereinafter, the compositions and the method according to the present invention will be explained in a more detailed manner.

### [Compositions]

The compositions according to the present invention may be a cosmetic composition, preferably a cosmetic composition for a keratinous substance, and more preferably a cosmetic composition for treating, hydrating, and/or cleansing acne-prove skin. The keratinous substance here means a material containing keratin as a main constituent element, and examples thereof include the facial or body skin, scalp, lips, and the like.

The form of the compositions according to the present invention is not particularly limited. In general, the composition according to the present invention can be liquid, semi-liquid, or solid at room temperature (25° C.) and atmospheric pressure (105 Pa). The composition may take various forms, such as a solution, an aqueous solution, a lotion, a milky lotion, a cream, a gel, a liquid gel, a paste, a serum, a suspension, a dispersion, a fluid, a milk, an emulsion (O/W or W/O form), or the like.

It is preferable that the compositions according to the present invention are in the form of a paste. Particularly, the compositions of the present invention have a unique wasabi-like paste texture.

The compositions according to the present invention may preferably be used as a cosmetic composition. The composition according to the present invention may be intended for application onto a keratinous substance, preferably skin, and in particular facial skin.

In some particular embodiments, the wasabi-like paste may be used as a 3-in-1 composition for acne-prone skin, which includes a daily skin clearing cleanser to be mixed with water, an overnight drying spot treatment in its original form, and/or a mask when layered on damped face.

The compositions can be used as a daily skin clearing cleanser, to be mixed with water, applied and rubbed on to the skin, and, after a short period of time, rinsed off with water. For spot treatment, the compositions can be dotted onto skin in its original form and rinsed off with water after a prolonged period of time, e.g., overnight. In the form of a mask, the compositions can be layered on damped face and retained on the skin for a selected period of time, then rinsed off water.

In one embodiment, the composition according to the present invention comprises (a) at least one glycolipid; (b) at least one salicylic acid or its derivatives according to formula (I); (c) at least one cationic polymer, (d) at least a clay, and (e) cosmetically acceptable solvents comprising water, and comprising glycerin present from about 2% to about 40%, relative to the total weight of the composition; and
wherein the pH of the composition is from about 3.5 to about 6.5.

In another embodiment, the composition according to the present invention comprises: (a) at least one rhamnolipid present from 2% by weight to 5% by weight, relative to the total weight of the composition; (b) at least one salicylic acid or its derivatives present from about 1% by weight to about 3% by weight, relative to the total weight of the composition; (c) at least one cationic polymer comprising chitosan; (d) at least one clay comprising kaolin; (e) cosmetically acceptable solvents comprising water, and comprising glycerin present from about 10% to about 30% by weight, relative to the total weight of the composition; wherein the pH of the composition is from about 3.5 to about 6.5; wherein the composition is suitable for 3-in-1 paste formulas for acne-prone skin as a skin cleanser, as a spot treatment, and as a mask.

The ingredients in the composition will be described in a detailed manner below.

### (Glycolipid)

The compositions according to the invention comprise one or more glycolipids. The amount of the glycolipid(s), including rhamnolipids, may be 0.10% to 10% by weight, preferably 1% to 7%, including 1%, 2%, 3%, 4% 5%, 6%, and 7%, by weight, more preferably 2% to 5% by weight, and in particular 3% by weight, relative to the total weight of the composition.

The term “glycolipid” is understood as meaning a compound formed from a lipid to which are attached one or more sugar compounds.

The one or more glycolipids may be selected from rhamnolipids, sophorolipids, glucolipids, trehalolipids, cellobiose lipids and mixtures thereof.

The one or more glycolipids are preferably selected from rhamnolipids, sophorolipids and mixtures thereof, more preferably rhamnolipids.

### Glucolipids:

The one or more glycolipids may be glucolipids, which contain a glucose moiety and can be represented by the general formula (I):

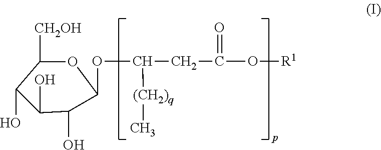

in which:
R¹ represents a hydrogen atom or a cation,
p denotes an integer ranging from 1 to 4, and
q denotes an integer ranging from 4 to 10, preferably equal to 6.

The glucolipids can be produced by the bacterium Alcaligenes sp. MM1. The appropriate fermentation methods are reviewed by M. Schmidt in his doctoral thesis (1990), Technical University of Braunschweig, and by Schulz et al. (1991) Z. Naturforsch., 46C, 197-203. The glucolipids are recovered from the fermentation broth by solvent extraction using diethyl ether or a dichloromethane:methanol or chloroform:methanol mixture.

### Sophorolipids:

The one or more glycolipids may be sophorolipids, which contain a sophorose moiety and can be represented by the general formula (II):

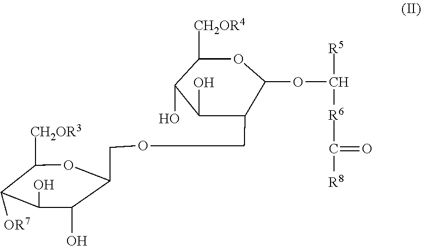

in which:
   R³ and R⁴ individually represent a hydrogen atom or an acetyl group,
   R⁵ represents a saturated or unsaturated, hydroxylated or non-hydroxylated hydrocarbon group having from 1 to 9 carbon atoms, preferably methyl,
   R⁶ represents a saturated or unsaturated, hydroxylated or non-hydroxylated hydrocarbon group having from 1 to 19 carbon atoms, with the proviso that the total number of carbon atoms in the groups R⁵ and R⁶ does not exceed 20 and is preferably from 14 to 18.

Sophorolipids may be incorporated into the composition according to the invention either in the form of the open-chain free acid, where R⁷ represents a hydrogen atom and R⁸ represents a hydroxy group OH, or in its lactone form, where a lactone ring is formed between R⁷ and R⁸, as indicated by formula (III):

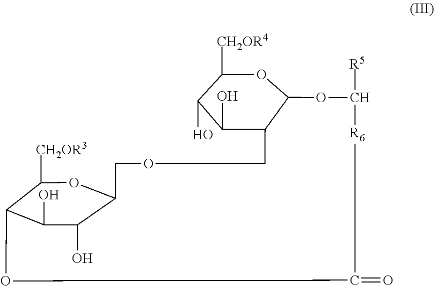

in which:
   R³, R⁴, R⁵ and R⁶ are as defined above,
with the proviso that at least one of R and R⁴ represents an acetyl group.

The sophorolipids can be produced by yeast cells, for example Torulopsis apicola and Torulopsis bombicola cells. The fermentation process generally uses sugars and alkanes as substrates.

Appropriate fermentation methods are reviewed in A. P. Tulloch, J. F. T. Spencer and P. A. J. Gorin, Can. J. Chem. (1962), 40, 1326, and U. Gobbert, S. Lang and F. Wagner, Biotechnology Letters (1984), 6 (4), 225. The resulting product is a mixture of various open-chain sophorolipids and of sophorolipid lactones that may be used in the form of mixtures, or the required form may be isolated.

It is possible to use as sophorolipids for example that sold under the Sopholiance S name by Givaudan and that sold under the BioToLife name by BASF.

### Trehalolipids:

The one or more glycolipids may be trehalolipids, which contain a trehalose fragment and can be represented by the general formula (IV):

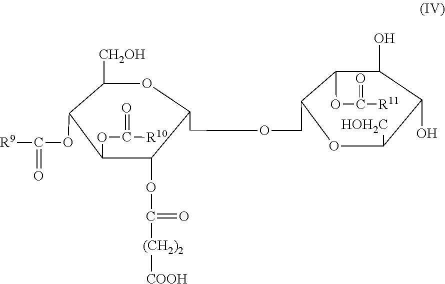

in which:
   R⁹, R¹⁰ and R¹¹ individually represent a saturated or unsaturated, hydroxylated or non-hydroxylated hydrocarbon radical having from 5 to 13 carbon atoms.

The trehalolipids can be produced by bacterial fermentation using the marine bacterium Arthrobacter sp. Ek 1 or the freshwater bacterium Rhodococcus erythropolis. Appropriate fermentation methods are provided by Ishigami et al. (1987), J. Jpn. Oil Chem. Soc., 36, 847-851, Schultz et al. (1991), Z. Naturforsch., 46C, 197-203, and Passeri et al. (1991), Z. Naturforsch., 46C, 204-209.

### Cellobiose Lipids:

The one or more glycolipids may be cellobiose lipids, which contain a cellobiose fragment and can be represented by the general formula (V):

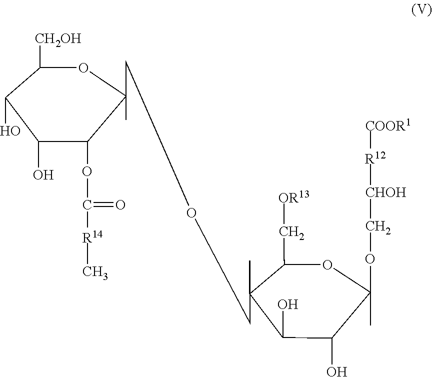

in which:
   R¹ represents a hydrogen atom or a cation,
   R¹² represents a saturated or unsaturated, hydroxylated or non-hydroxylated hydrocarbon radical having from 9 to 15 carbon atoms, preferably 13 carbon atoms,
   R¹³ represents a hydrogen atom or a acetyl group;
   R¹⁴ represents a saturated or unsaturated, hydroxylated or non-hydroxylated hydrocarbon radical having from 4 to 16 carbon atoms.

The cellobiose lipids can be produced by cells of fungi of the genus Ustilago. Appropriate fermentation processes are provided by Frautz, Lang and Wagner (1986), Biotech. Letts., 8, 757-762.

### Rhamnolipids:

The one or more glycolipids may be rhamnolipids.

The composition according to the invention preferably comprises one or more rhamnolipids.

Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of 3-hydroxylated fatty acids linked to one another by an ester bond.

More specifically, these mono-rhamnolipids and di-rhamnolipids correspond to the following formula (VI):

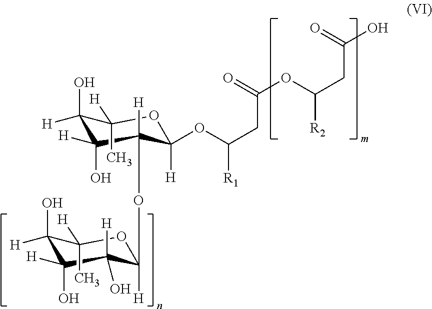

in which:
   m denotes an integer equal to 2, 1 or 0,
   n denotes an integer equal to 1 or 0, and
   R₁ and R₂, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical.

Thus, when n is equal to 0, the formula (VI) protects mono-rhamnolipids and, when n is equal to 1, it protects di-rhamnolipids.

The composition according to the invention preferably comprises at least one di-rhamnolipid.

The composition according to the invention preferably comprises at least one di-rhamnolipid of formula (VI) in which:
m denotes an integer equal to 2, 1 or 0;
n denotes an integer equal to 1; and
R₁ and R₂, each independently represent identical or different hydrocarbon radicals having from 2 to 24 carbon atoms, preferably from 5 to 13 carbon atoms, that are branched or unbranched, substituted or unsubstituted, in particular hydroxy-substituted, and saturated or unsaturated, preferably a singly, doubly or triply unsaturated alkyl radical, and also the salts thereof, solvates thereof and optical isomers thereof.

The glycosidic bond between the two rhamnose fragments may be in the alpha or beta configuration and is preferably in the alpha configuration.

In the context of the invention,
the salts of the di-rhamnolipids of formula (VI) are more particularly the carboxylate salts thereof with an organic or inorganic cation and especially with a cation selected from sodium, potassium, calcium and ammonium.
the solvated forms of the di-rhamnolipids of formula (VI) are more particularly those solvated with one or more molecules of water or of organic solvents, for example a hydrate or a solvate of a linear or branched alcohol, such as ethanol or isopropanol, the optically active carbon atoms of the fatty acids preferably being in the form of the R enantiomers, and
the term “alkyl” radical denotes a saturated, linear or branched aliphatic group; for example, a C₁-C₂₀ alkyl group having a linear or branched hydrocarbon chain of 1 to 20 carbon atoms, more particularly a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

The composition according to the invention preferably comprises at least one di-rhamnolipid of formula (VI) in which:
m denotes an integer equal to 2, 1 or 0;
n denotes an integer equal to 1; and
R¹ and R², which are identical or different, are selected from pentenyl, hexenyl, heptenyl, octenyl, nonenyl, decenyl, undecenyl, dodecenyl and tridecenyl radicals and radicals of formula —(CH₂)ₒCH₃, with o denoting an integer ranging from 1 to 23, in particular from 3 to 15 and more particularly from 4 to 12.

According to one embodiment of the invention, the composition according to the invention comprises at least one di-rhamnolipids of general formula (VI) in which m is equal to 1

According to one embodiment of the invention, the composition according to the invention comprises a mixture of at least two, preferably at least three, di-rhamnolipids of general formula (VI) in which m is preferably equal to 1.

According to another embodiment of the invention, the composition according to the invention comprises a mixture comprising at least one mono-rhamnolipid.

More preferably, the composition according to the invention comprises at least one dirhamnolipid of the following formula (VII):

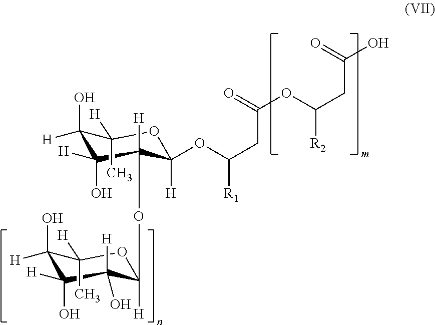

in which:
   m denotes an integer equal to 2, 1 or 0; preferably, m is equal to 1,
   n denotes an integer equal to 1,
   R¹ is a —(CH₂)ₚ—CH₃ radical, with p being an integer varying from 1 to 23, preferably from 4 to 12,
   R² is a —(CH₂)_{q}—CH₃ radical, with q being an integer varying from 1 to 23, preferably from 4 to 12, and also the salts thereof, solvates thereof and optical isomers thereof.

By way of illustration and without limiting the di-rhamnolipids of formula (VII) that may be suitable for the invention, mention may be made in particular of the compounds of formula di-RL-CXCY, such as are defined in Table 1 below.

The formula di-RL-CXCY is an alternative way of writing in order to represent a di-rhamnolipid (di-RL) functionalized by two radicals R¹ and R² respectively represented by the symbols CX and CY, the integers X and Y being respectively equal to p+4 and q+4.

TABLE 1

| Composés | Di-RL-CXCY | p | q |
|---|---|---|---|
| 1 | diRL-C8C8 | 4 | 4 |
| 2 | diRL-C8C10 | 4 | 6 |
| 3 | diRL-C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

According to a preferred embodiment, the composition according to the invention comprises at least one di-rhamnolipid of formula (VII) in which p and q are identical and equal to 6 and m is equal to 1, also referred to as di-RL-C10C10, or one of the salts, solvates and optical isomers thereof.

Preferably, the di-rhamnolipid of formula (VII) in which p and q are identical and equal to 6 and m is equal to 1 is present in the composition according to the invention in a proportion of at least 50% by weight and preferably of from 51% to 85% by weight, relative to the total weight of rhamnolipids.

According to another embodiment, the composition according to the invention comprises at least one di-rhamnolipid of formula (VII) in which m is equal to 1, p is equal to 6 and q is equal to 8.

According to another embodiment, the composition according to the invention comprises at least one di-rhamnolipid of formula(VI) in which n and m are equal to 1, R₁ represents a —(CH₂)ₒCH₃ radical, with o being an integer varying from 4 to 12, and R² is selected from the pentenyl, hexenyl, heptenyl, octenyl, nonenyl, decenyl, undecenyl, dodecenyl and tridecenyl radicals; preferably, R¹ represents a —(CH₂)₆CH₃ radical and R² a nonenyl radical.

According to another preferred embodiment, the composition according to the invention comprises a mixture of at least two, in particular at least three, di-rhamnolipids of formula (VI) or of formula (VII) selected from:
a di-rhamnolipid of formula (VII) in which p and q are identical and equal to 6 and m is equal to 1;
a di-rhamnolipid of formula (VII) in which m is equal to 1, p is equal to 6 and q is equal to 8; and
at least one di-rhamnolipid of formula (VI) in which n and m are equal to 1, R₁ represents a —(CH₂)ₒCH₃ radical, with o being an integer varying from 4 to 12, and R² is selected from the pentenyl, hexenyl, heptenyl, octenyl, nonenyl, decenyl, undecenyl, dodecenyl and tridecenyl radicals; preferably, R¹ represents a —(CH₂)₆CH₃ radical and R² a nonenyl radical.

Preferably, the composition according to the invention comprises a mixture of at least two, in particular at least three, di-rhamnolipids of formula (VI) or of formula (VII) selected from:
at least 50% by weight and preferably of from 51% to 85% by weight of a di-rhamnolipid of formula (VII) in which p and q are identical and equal to 6 and m is equal to 1, relative to the total weight of rhamnolipids.
from 0.5% to 25% by weight, preferably from 5% to 15% by weight, of a dirhamnolipid of formula (VII) in which p is equal to 6, q is equal to 8 and m is equal to 1, relative to the total weight of rhamnolipids, and
from 0.5% to 15% by weight, preferably from 3% to 12% by weight, preferably from 5% to 10% by weight, of a dirhamnolipid of formula (VI) in which n and m are equal to 1, R¹ represents a —(CH₂)₆CH₃ radical and R² represents a nonenyl radical, relative to the total weight of rhamnolipids.

As specified above, rhamnolipids are customarily prepared by processes known to those skilled in the art starting from bacterial producers, such as Pseudomonas.

Appropriate fermentation methods are reviewed by D. Haferburg, R. Hommel, R. Claus and H. P. Kleber in Adv. Biochem. Ing./Biotechnol. (1986), 33, 53-90, and by F. Wagner, H. Bock and A. Kretschmar in Fermentation (ed. R. M. Lafferty) (1981), 181-192, Springer Verlag, Vienna.

Use may be made, as rhamnolipid, of the one sold under the name Rheance One by Evonik (INCI name: glycolipids).

The amount of rhamnolipids, may be 0.10% to 10% by weight, preferably 10% to 7%, including 1%, 2%, 3%, 4% 5%, 6%, and 7%, by weight, more preferably 2% to 5% by weight, and in particular 3% by weight, relative to the total weight of the composition.

### (Salicylic Acid or its Derivatives)

The composition according to the present invention comprises (b) at least one salicylic acid or its derivatives according to formula (I). A single type of the salicylic acid or its derivative may be used, but two or more different types of the salicylic acid or its derivatives may be used in combination.

The salicylic derivatives in accordance with the present invention correspond to formula (I):

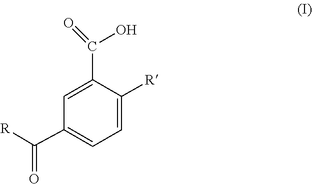

in which:
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

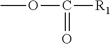

in which R₁ denotes a linear or branched, saturated or unsaturated aliphatic chain containing 1 to 18 carbon atoms;
and also salts thereof derived from an inorganic or organic base.

Preferably, the R radical in formula (I) denotes a linear, branched or cyclic, saturated aliphatic chain containing from 3 to 11 carbon atoms; an unsaturated chain containing from 3 to 17 carbon atoms and comprising one or more conjugated or unconjugated double bonds; it being possible for said hydrocarbon-based chains to be substituted with one or more substituents, which may be identical or different, chosen from halogen atoms, a trifluoromethyl group, hydroxyl groups in free form or esterified with an acid containing from 1 to 6 carbon atoms, or a carboxyl function in free form or esterified with a lower alcohol containing from 1 to 6 carbon atoms.

In one preferred embodiment, the R radical in formula (I) denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms.

Preferentially, R′ in formula (I) is a hydroxyl group or an ester group of formula:

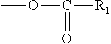

in which R¹ denotes a radical —(CH₂)ₙ—CH₃ where n is a number ranging from 0 to 14.

The salicylic acid derivatives that are more particularly preferred are those according to formula (I) in which the R radical is a C₃-C₁₀ alkyl group and/or R′ denotes hydroxyl.

Other particularly advantageous compounds are those in which R represents a chain derived from caprylic, linoleic, linolenic or oleic acid.

Another group of particularly preferred salicylic acid derivatives is constituted of compounds in which the R radical denotes a C₃-C₁₀ alkyl group bearing a carboxyl function in free form or esterified with a lower alcohol containing from 1 to 6 carbon atoms and R′ denotes hydroxyl.

The salicylic acid or its derivatives of formula (I) that may be used according to the present invention are in particular described in U.S. Pat. Nos. 6,159,479 and 5,558,871, FR 2,581,542, U.S. Pat. No. 4,767,750, EP 378 936, U.S. Pat. Nos. 5,267,407, 5,667,789, 5,580,549 and EP-A-570,230.

Among the particularly preferred salicylic acid derivatives of formula (I), mention may be made of 5-n-octanoylsalicylic acid (or capryloyl salicylic acid); 5-n-decanoylsalicylic acid; 5-n-dodecanoylsalicylic acid; 5-n-heptyloxysalicylic acid, and the corresponding salts thereof. The derivative in question is preferably 5-n-octanoylsalicylic acid.

For the purposes of the present invention, the salts of the salicylic acid or its derivatives are also considered. As the salts derived from inorganic bases, mention may particularly be made of those derived from alkali metal or alkaline-earth metal hydroxylated bases, for instance sodium hydroxide or potassium hydroxide, and ammonia. As regards the salts derived from the organic bases, mention may particularly be made of those derived from bases of amine or alkanolamine type.

The amount of the salicylic acid or its derivatives may be from 0.1% to 5% by weight, preferably from 1% to 3% by weight, most preferably 2% by weight, relative to the total weight of the composition.

### (Cationic Polymer)

In various embodiments, the acne care composition may comprise at least one cationic polymer selected from nature-based polymers that are polysaccharides, and other natural (i.e., plant, animal, or bacterial based), synthetic, or modified cationic nature-based or synthetic polymers.

The at least one cationic polymer may be selected from the group consisting of chitosan, chitosan oligosaccharide, chitin, cyclodextrin, cationic gelatin, cationic dextran, cationic cellulose, polylysine, polyomithine, histone, collagen, chitosan-cysteine, chitosan-thiobutylamidine, chitosan-thioglycolic acid, and combinations thereof.

Examples of cationic polymers include polysaccharide-based delivery molecules (e.g., chitosan, cyclodextrin, cationic gelatin, cationic dextran, cationic cellulose), cationic peptides and their derivatives (e.g., polylysine, polyomithine), peptide/protein polymers ((e.g., histone, collagen), linear or branched synthetic polymers (e.g., polybrene, polyethyleneimine), natural polymers (e.g., histone, collagen), synthetic dendrimers, cationic thiolated biopolymers (nature-based thiomers or nature-based dendrimers, e.g., chitosan-cysteine, chitosan-thiobutylamidine as well as chitosan-thioglycolic acid).

In some embodiments, the composition may include or exclude any other cationic polymer, including a nature-based polymer or synthetic polymer.

In preferred embodiments, the cationic polymer may be a polysaccharide derived from β-linked D-glucosamine and N-acetyl-D-glucosamine. A particularly preferred cationic polymer is chitosan.

In various embodiments, chitosan has a molecular weight (MW) in a range from about 1 kDa to about 1000 kDa. In some particular embodiments, chitosan has a MW that is “low” and is in the range from about 1 kDa to about 20 kDa, or from about 10 kDa to about 20 kDa, or from about 12 kDa to about 18 kDa. In some embodiments, the chitosan has a Chitosan MW=˜27 kDa.

The amount of the chitosan may be from 0.01% to 3% by weight, preferably from 0.05% to 1% by weight, and most preferably 0.10% to 0.2% by weight, including but not limited to about 0.10%, 0.15%, and 0.2%, relative to the total weight of the composition.

According to some preferred embodiments, the compositions comprise chitosan present at about 0.10%, by weight, relative to the total weight of the composition.

### (Clay)

The at least one clay may be selected from the group of clays consisting of kaolinites, smectite, sodium smectite, calcium smectite, illite, chlorite, vermiculite, attapulgite, and combinations thereof.

In some particular embodiments, the at least one clay may be a smectite clay in the mica family of phylosilicates. And in some preferred embodiments, the at least one clay may comprise hectorite. Inmost embodiments, the at least one clay comprises kaolin.

The amount of the clay may be from 5% to 25% by weight, preferably from 10% to 20% % by weight, including 10%, 11%, 12%, 13%, 14%, 15%, 16%, 17%, 18% 19% and 20%; most preferably 15% by weight, relative to the total weight of the composition.

### (Cosmetically Acceptable Solvents)

Compositions according to the disclosure contain at least one cosmetically acceptable solvent. In various embodiments, the cosmetically acceptable solvent may be chosen from water.

### Water:

In accordance with the various embodiments, water is present in the composition in a range from about 10% to about 60%, or from about 20% to about 55%, or from about 25% to about 50%, or from about 28% to about 49%, or any suitable combination, sub-combination, range, or sub-range thereof by weight, based on the weight of the composition. One of ordinary skill in the art, however, will appreciate that other ranges are within the scope of the invention.

Thus, water may be present by weight, based on the weight of the composition, from about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to about 60 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene.

The water phase may also comprise reconstituted thermal water, that is to say a water comprising trace elements such as zinc, copper, magnesium, etc., reconstituting the characteristics of a thermal water.

### Water-Soluble Solvents:

In accordance with some embodiments, the compositions include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions.

### Glycerin:

In some preferred embodiments, the water-soluble solvent includes glycerin.

The amount of the glycerin may be from 1% to 45% by weight, preferably from 2% to 40% % by weight, most preferably 10% to 30% by weight, including but not limited to 10%, 15%, 20%, 25%, and 30%, relative to the total weight of the composition.

As examples of organic solvents, non-limiting mentions can be made of ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols, glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, 1,2-hexanediol, 1,2-pentanediol, and 4-methyl-1,2-pentanediol.

Further non-limiting examples of water-soluble solvents include alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, tiacetine, sulfolane, or mixtures thereof.

In accordance with the various embodiments the amount of the at least one water-soluble solvent is from about 0.1% to about 25%, or from about 0.1% to about 2%, or from about 0.1% to about 1T %, or from about 0.1% to about 0.8%, or from about 0.1% to about 0.50%, or from about 1% to about 20%, or from about 1% to about 10%, or from about 2% to about 8%, or any suitable combination, sub-combination, range, or sub-range thereof by weight, based on the weight of the composition. One of ordinary skill in the art, however, will appreciate that other ranges are within the scope of the invention.

In some embodiments, the compositions include more than one water soluble solvent, each water soluble solvent presents in an amount as set forth herein above, wherein each different water soluble solvent may be present within one of the ranges selected from the ranges set forth herein above.

Thus, water-soluble solvents may be present by weight, based on the total weight of the composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### (Surfactants)

In various embodiments, the compositions further comprise the surfactant sodium cocoyl isethionate.

In some embodiments, the compositions may include, or alternatively may be substantially free from, or exclude any one or more of nonionic, cationic, or anionic surfactants. In some embodiments, the compositions exclude all surfactants except sodium cocoyl isethionate.

In some embodiments, the compositions also include at least one additional surfactant selected from glyceryl oleate, glyceryl stearate, and cetyl alcohol. In some embodiments, the composition includes at least glyceryl stearate SE in addition to sodium cocoyl isethionate.

The concentration of sodium cocoyl isethionate present in the compositions is from about 1% to about 20%, or from about 2% to about 18%, or from about 5% to about 15%, or from about 8% to about 12%, or about 10%, or any suitable combination, sub-combination, range, or sub-range thereof by weight, based on the weight of the composition. One of ordinary skill in the art, however, will appreciate that other ranges are within the scope of the invention.

Thus, sodium cocoyl isethionate is present, by weight, based on the total weight of the composition, from about 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17, 18, 19 to about 20 weight percent, including increments and ranges therein and there between.

When present, an additional surfactant, for example, glyceryl stearate SE, is present in the compositions is from about 1% to about 20%, or from about 2% to about 18%, or from about 5% to about 15%, or from about 8% to about 12%, or from about 2% to about 6%, or about 5%, or any suitable combination, sub-combination, range, or sub-range thereof by weight, based on the weight of the composition. One of ordinary skill in the art, however, will appreciate that other ranges are within the scope of the invention.

Thus, an additional surfactant, when present, is present, by weight, based on the total weight of the composition, from about 1.0, 1.1, 1.2, 1.3, 1.4, 1.5, 1.6, 1.7, 1.8, 1.9, 2.0, 3, 4, 5, 6, 7, 8, 9, 10, 11, 12, 13, 14, 15, 16, 17, 18, 19 to about 20 weight percent, including increments and ranges therein and there between.

### (Additives)

In some embodiments, there may be one or more optional actives or other ingredients (herein, “additives”) present in the compositions, the one or more additives optionally selected from: anti-microbials; chelating agents; oils; fillers; penetrants; sequestrants; fragrances; dispersants; skin care actives such as ceramides, for example, sodium lauroyl lactylate (and) ceramide np (and) ceramide ap (and) phytosphingosine (and) cholesterol (and) xanthan gum (and) carbomer (and) ceramide eop; opacifiers; alpha-hydroxy acids; organic and inorganic UV filters; citric acid; phenylethyl resorcinol; hydroxypropyl tetrahydropyrantriol; hydroxyacetophenone; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus pinaster bark/bud extract), ellagic acid; hyaluronic acid and its derivatives; escin (also known as Aescin, a mixture of saponins with anti-inflammatory, vasoconstrictor and vasoprotective effects found in Aesculus hippocastanum); retinol; niacinamide; and vitamins and vitamin derivatives, such as tocopherol and ascorbic acid; and combinations thereof.

Although the aforementioned optional additives are given as an example, it will be appreciated that other optional components compatible with cosmetic applications known in the art may be used. And of course, any one of the aforementioned additives may be excluded.

In some embodiments, the compositions include at least one additive selected from the group consisting of sodium hydroxide, niacinamide, lactic acid, cetyl alcohol, trisodium ethylenediamine disuccinate, ceramides, and a combination thereof.

In some embodiments, the composition includes additives comprising sodium hydroxide (0.3-0.5%), niacinamide (2%), lactic acid (0.05%), cetyl alcohol (2.5%), trisodium ethylenediamine disuccinate (0.5-1%), and ceramides (sodium lauroyl lactylate (and) ceramide NP (and) ceramide AP (and) phytosphingosine (and) cholesterol (and) xanthan gum (and) carbomer (and) ceramide EOP) (*final active concentration 0.25%)).

### Niacinamide:

The compositions according to the present invention may comprise niacinamide present at about from about 0.1 to about 10% by weight, preferably from about 0.5% to 5% by weight, most preferably from about 1% to about 3% by weight, including but not limited to 1%, 2%, and 3% by weight, and most preferably about 2% by weight, relative to the total weight of the composition.

In accordance with the various embodiments, the amounts of additives, for example, actives and other components, that may be present in the compositions can range from about 0.001% to about 50%, or from about 0.5% to about 30%, or from about 1.5% to about 20%, and from about 5% to about 15%, or any suitable combination, sub-combination, range, or sub-range thereof by weight, based on the weight of the acne care composition.

Thus, one or a combination of additives may be present in the compositions, by weight, based on the weight of the composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

### (Thickener)

In various embodiments, the compositions may comprise at least one stabilizer and/or thickening agent (thickener). The thickener may be thickening polymers. Non-limiting examples of thickening polymers include sclerotium gum, xanthan gum, carrageenan, acacia, agar, algin, alginic acid, ammonium alginate, amylopectin, calcium alginate, sodium alginate, calcium carrageenan, camitine, carrageenan, dextrin, gelatin, gellan gum, guar gum, tragacanth gum, acacia gum, Arabic gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sodium carboxymethyl dextran, sodium carrageenan, tragacanth gum, xanthan gum, modified xanthan gum, biosacharide gum, chitin, levan, elsinan, collagen, gelatin, zein, gluten, soy protein, casein, and mixtures thereof.

According to some embodiments, the composition comprises one or more of carrageenan, xanthan gum, and sclerotium gum, or a combination thereof, present in a range from about 0.10% to about 5%, or at about 1%, by weight of the composition.

### Carrageenan:

According to some embodiments, the acne care composition comprises carrageenan as a particularly preferred stabilizer/thickening agent, present at about 1%, by weight of the composition.

The at least one thickener, when present, is present in the composition from about 0.10% to about 5%, or from about 0.2% to about 3%, or from about 0.5% to about 2%, or about 1%, by weight of the composition, or any suitable combination, sub-combination, range, or sub-range thereof by weight, based on the weight of the acne care composition. One of ordinary skill in the art, however, will appreciate that other ranges are within the scope of the invention.

Thus, the at least one thickener may be present, by weight, based on the total weight of the composition, from about 0.05, 0.06, 0.07, 0.08, 0.90, 0.1, 0.2, 0.3, 0.4, 0.5, 0.6, 0.7, 0.8, 0.9, 1.0, 2, 3, 4 to about 5 weight percent, including increments and ranges therein and there between.

### (pH Adjusting Agent)

The pH of the composition according to the present invention may be adjusted to the desired value using at least one pH adjusting agent, such as an acidifying or a basifying agent, for example, which are commonly used in cosmetic products.

The pH of the composition is from about 3.5 to about 6.5, preferably from about 4 to 6, most preferably from about 4.5 to 5.5, and particularly preferred pH is about 5.5 or about 5.0.

Among the acidifying agents, mention may be made, by way of example, of mineral or organic acids such as hydrochloric acid, ortho-phosphoric acid, sulfuric acid, carboxylic acids such as acetic acid, tartaric acid, citric acid, and lactic acid, and sulfonic acids.

Among the basifying agents, mention may be made, by way of example, of hydroxides of an alkali metal or an alkaline-earth metal, for instance sodium hydroxide or potassium hydroxide; quaternary ammonium hydroxides and guanidinium hydroxide; alkali metal silicates, such as sodium metasilicates; amino acids, preferably basic amino acids, such as arginine, lysine, omithine, citrulline and histidine; carbonates and bicarbonates, particularly of a primary amine, secondary amine or tertiary amine, of an alkali metal or alkaline-earth metal, or of ammonium; and the compounds of the following formula:

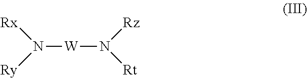

in which
W is a C₁-C₆ alkylene residue optionally substituted with a hydroxyl group or a C₁-C₆ alkyl group; Rx, Ry, Rz and Rt, which may be identical or different, represent a hydrogen atom or a C₁-C₆ alkyl, C₁-C₆ hydroxyalkyl or C₁-C₆ aminoalkyl group. Mention may especially be made of 1,3-diaminopropane, 1,3-diamino-2-propanol, spermine and spermidine.

The pH adjusting agent(s) may be used in an amount ranging from 0.001% to 10% by weight, preferably from 0.01% to 5% by weight, and more preferably from 0.1% to 3% by weight, relative to the total weight of the composition.

The composition according to the present invention can be prepared by mixing the above-described essential and optional ingredients in a conventional manner. In the case that at least one of the above ingredients is solid at room temperature, the ingredient can be heated until it is dissolved. It is possible to further comprise mixing any of the optional ingredients and heating the composition until an ingredient is dissolved.

[Method for Treating, Hydrating and/or Cleansing Acne-Prone Skin]

The present invention also relates to a method for treating, hydrating, and/or cleansing acne-prone skin including scalp, lips, in particular facial skin, comprising: applying onto the acne-prone skin a composition comprising:
(a) at least one glycolipid;
(b) at least one salicylic acid or its derivatives of formula (I) above;
(c) at least one cationic polymer selected from a polysaccharide derived from 13-linked D-glucosamine or N-acetyl-D-glucosamine; and
(d) at least one clay;
(e) cosmetically acceptable solvents comprising water, and comprising glycerin present from about 2% to about 40% by weight, relative to the total weight of the composition; and
   wherein the pH of the composition is from about 3.5 to about 6.5.

Treating acne-prone skin in present invention means reduction of sebum, or deposition of salicylic acid or its derivatives, or reduction of viable cells of Cutibacterium acnes.

Cleanability of acne-prone skin is calculated as below:

$$Cleansability = \frac{{De}_{sample cleaned} - {De}_{black reference}}{{De}_{clean reference} - {De}_{black reference}}$$

$${D}_{e} = \sqrt{{D}_{l}^{2} + {D}_{a}^{2} + {D}_{b}^{2}}$$

De: color difference in a rectangular coordinate system
Black reference: bioskin with sebollution applied following pore deep clean protocol
Clean reference: clean bioskin w/o any sebollution

The composition is generally applied on acne-prone skin with hands, fingers, or an applicator. The present invention may comprise an optional step of rinsing the composition from the keratinous substance after it is applied.

The composition used in the method according to the present invention may include any of the optional ingredients as explained above for the composition according to the present invention.

### EXAMPLES

The present invention will be described in more detail by way of examples which however should not be construed as limiting the scope of the present invention.

### Examples 1 to 11 of Present Invention

Compositions of Examples 1 to 11 were prepared by mixing the raw materials as listed in Table 1 with a magnetic stirrer. The numerical values for the amounts of the raw materials shown in Table 1 are all based on “% by weight.” Note that rhamnolipids were obtained from EVONIK (trade name: RHEANCE One, INCI name: glycolipids), which contained about 50% by weight of rhamnolipids in water. Thus, the weight percentages of rhamnolipids in the entire application including in the Examples, Specification, and Claims refer to the weight percentages of the rhamnolipids aqueous solution.

### [Hydration Study Results]

Examples 8-11 were evaluated against petroleum jelly (positive control) and bare skin for hydration. Subjects cleansed each volar forearm with one 70% isopropyl alcohol wipe. Skin was permitted to air dry for thirty (30) minutes prior to the baseline measurements, with the volar forearms exposed in a room with controlled temperature and humidity conditions. Baseline measurements of each site were taken with the Comeometer 825. Additional measurements were taken at 15 minutes and one hour.

TABLE 1

| Ingredient | Function | Ex. 1 | Ex. 2 | Ex. 3 | Ex. 4 | Ex. 5 | Ex. 6 | Ex. 7 | Ex. 8 | Ex. 9 | Ex. 10 | Ex. 11 |
|---|---|---|---|---|---|---|---|---|---|---|---|---|
| Sodium Hydroxide | Active | 0.350 | 0.350 | 0.300 | 0.300 | 0.300 | 0.300 | 0.350 | 0.450 | 0.450 | 0.250 | 0.450 |
| | Compound | | | | | | | | | | | |
| Tartaric Acid | Active | | | | 0.200 | | | | | | | |
| | Compound | | | | | | | | | | | |
| Lactic Acid | Active | 0.050 | 0.050 | | | 0.050 | 0.050 | 0.050 | 0.050 | 0.050 | 0.050 | 0.050 |
| | Compound | | | | | | | | | | | |
| Trisodium | Active | 0.500 | 0.500 | 0.500 | | 0.500 | 0.500 | 0.500 | 0.500 | 0.500 | 0.500 | 0.500 |
| Ethylenediamine | Compound | | | | | | | | | | | |
| Disuccinate | | | | | | | | | | | | |
| Sodium Lauroyl | Active | 0.250 | 0.250 | 0.250 | 0.250 | 0.250 | 0.250 | 0.250 | 0.250 | 0.250 | 0.250 | 0.250 |
| Lactylate (and) | Compound | | | | | | | | | | | |
| Mixture of | | | | | | | | | | | | |
| Ceramides (and) | | | | | | | | | | | | |
| Phytosphingosine | | | | | | | | | | | | |
| (and) Cholesterol | | | | | | | | | | | | |
| (and) Xanthan Gum | | | | | | | | | | | | |
| (and) Carbomer | | | | | | | | | | | | |
| Cetyl Alcohol | Fatty | 2.500 | 2.500 | 2.500 | 2.500 | 2.500 | 2.500 | 2.500 | 2.500 | 2.500 | 2.500 | 2.500 |
| | Compound | | | | | | | | | | | |
| Kaolin | Filler | 15.0 | 15.0 | 15.0 | 15.0 | 15.0 | 15.0 | 15.0 | 10.0 | 15.0 | 15.0 | 15.0 |
| Carrageenan | Polymer | 1.0 | 1.0 | 1.0 | 1.0 | 1.0 | 1.0 | 1.0 | 0.8 | 1.0 | 1.0 | 1.0 |
| Chitosan | Polymer | 0.10 | 0.10 | 0.10 | 0.10 | 0.10 | 0.10 | 0.10 | 0.10 | 0.10 | 0.10 | 0.10 |
| Salicylic Acid | Active | 2.0 | 2.0 | 2.0 | 2.0 | 2.0 | 2.0 | 2.0 | 2.0 | 2.0 | 2.0 | 2.0 |
| Phenoxyethanol | Preservative | 0.30 | | | | | | 0.30 | | | | |
| Water | Solvent | 55.95 | 51.25 | 56.35 | 56.65 | 51.30 | 56.30 | 55.95 | 23.35 | 48.15 | 48.35 | 28.15 |
| Glycerin | Solvent | 2.0 | 2.0 | 2.0 | 2.0 | 2.0 | 2.0 | 2.0 | 40.0 | 10.0 | 10.0 | 30.0 |
| Glyceryl Stearate SE | Surfactant | 5.0 | 5.0 | 5.0 | 5.0 | 5.0 | 5.0 | 5.0 | 5.0 | 5.0 | 5.0 | 5.0 |
| Sodium Cocoyl | Surfactant | | 10.0 | | | 10.0 | 10.0 | | 10.0 | 10.0 | 10.0 | 10.0 |
| Isethionate | | | | | | | | | | | | |
| Sodium Methyl | Surfactant | 10.0 | 5.0 | 10.0 | 10.0 | 5.0 | | 10.0 | | | | |
| Cocoyl Taurate | | | | | | | | | | | | |
| Rhamnolipids | Surfactant | 3.0 | 3.0 | 3.0 | 3.0 | 3.0 | 3.0 | 3.0 | 3.0 | 3.0 | 3.0 | 3.0 |
| Niacinamide | Active | 2.0 | 2.0 | 2.0 | 2.0 | 2.0 | 2.0 | 2.0 | 2.0 | 2.0 | 2.0 | 2.0 |

The results of the hydration study are provided in Table 2, with the values indicating the level of hydration at baseline, 15 minutes, and 1 hour. As shown in Table 2, Examples 8 through 11 were effective for hydrating the skin at 15 minutes and one-hour intervals, compared to the baseline as well as evaluated against petroleum jelly (positive control) and bare skin.

TABLE 2

| Example | Baseline | 15 min | 1 hr |
|---|---|---|---|
| Petroleum Jelly | 31.47 ± 4.60 | 37.00 ± 9.34 | 39.36 ± 8.06 |
| Bare (Wet) | 31.92 ± 4.82 | 30.37 ± 7.10 | 30.88 ± 7.09 |
| Ex. 8 | 31.54 ± 5.40 | 45.47 ± 9.30 | 45.71 ± 10.23 |
| Ex. 10 | 30.12 ± 5.36 | 32.28 ± 7.64 | 32.83 ± 7.70 |
| Ex. 9 | 29.16 ± 4.94 | 36.07 ± 8.14 | 35.38 ± 8.35 |
| Ex. 11 | 30.28 ± 4.98 | 37.58 ± 7.42 | 38.37 ± 8.78 |

### Examples 12 to 19 of Present Invention

Compositions of Examples 12 to 19 were prepared by mixing the raw materials as listed in Table 3 with a magnetic stirrer. The numerical values for the amounts of the raw materials shown in Table 3 are all based on “% by weight.” Note that rhamnolipids were obtained from EVONIK (trade name: RHEANCE One, INCI name: glycolipids), which contained about 50% by weight of rhamnolipids in water. Thus, the weight percentages of rhamnolipids in the entire application including in the Examples, Specification, and Claims refer to the weight percentages of the rhamnolipids aqueous solution.

TABLE 3

| INCI name | Cosmetic type | Ex 12 | Ex 13 | Ex 14 | Ex 15 | Ex 16 | Ex 17 | Ex 18 | Ex 19 |
|---|---|---|---|---|---|---|---|---|---|
| sodium hydroxide | active compound | 0.45 | 0.40 | 0.45 | 0.35 | 0.30 | 0.30 | 0.45 | 0.45 |
| niacinamide | active compound | 2.00 | 2.00 | 2.00 | 2.00 | 2.00 | 2.00 | 2.00 | 2.00 |
| glyceryl stearatese | surfactant | 5.00 | 5.00 | 5.00 | 5.00 | 5.00 | 5.00 | 5.00 | 5.00 |
| carrageenan | polymer | 1.00 | 1.00 | 1.00 | 1.00 | 1.00 | 1.00 | 0.80 | 1.00 |
| salicylic acid | active compound | 2.00 | 2.00 | 2.00 | 2.00 | 2.00 | 2.00 | 2.00 | 2.00 |
| phenoxyethanol | preservative | | | | 0.30 | | | | |
| water | solvent | 28.15 | 47.70 | 48.15 | 55.95 | 51.30 | 56.30 | 23.35 | 38.15 |
| glycerin | solvent | 30.00 | 10.00 | 10.00 | 2.00 | 2.00 | 2.00 | 40.00 | 20.00 |
| lactic acid | active compound | 0.05 | 0.05 | 0.05 | 0.05 | 0.05 | 0.05 | 0.05 | 0.05 |
| cetyl alcohol | fatty compound | 2.50 | 2.50 | 2.50 | 2.50 | 2.50 | 2.50 | 2.50 | 2.50 |
| kaolin | filler | 15.00 | 15.00 | 15.00 | 15.00 | 15.00 | 15.00 | 10.00 | 15.00 |
| sodium cocoyl | surfactant | 10.00 | 10.00 | 10.00 | | 10.00 | 10.00 | 10.00 | 10.00 |
| isethionate | | | | | | | | | |
| trisodium | active compound | 0.50 | 1.00 | 0.50 | 0.50 | 0.50 | 0.50 | 0.50 | 0.50 |
| ethylenediamine | | | | | | | | | |
| disuccinate | | | | | | | | | |
| sodium lauroyl | active compound | 0.25 | 0.25 | 0.25 | 0.25 | 0.25 | 0.25 | 0.25 | 0.25 |
| lactylate (and) | | | | | | | | | |
| ceramide np (and) | | | | | | | | | |
| ceramide ap (and) | | | | | | | | | |
| phytosphingosine | | | | | | | | | |
| (and) cholesterol | | | | | | | | | |
| (and) xanthan | | | | | | | | | |
| gum (and) | | | | | | | | | |
| carbomer (and) | | | | | | | | | |
| ceramide eop | | | | | | | | | |
| glycolipids | surfactant | 3.00 | 3.00 | 3.00 | 3.00 | 3.00 | 3.00 | 3.00 | 3.00 |
| chitosan | polymer | 0.10 | 0.10 | 0.10 | 0.10 | 0.10 | 0.10 | 0.10 | 0.10 |
| sodium methyl | surfactant | | | | 10.00 | 5.00 | | | |
| cocoyl taurate | | | | | | | | | |

### [Sebum Control Study Results]

Sebum control studies were conducted according to the following procedure:
1. Wash face with water & acclimatization in standard condition for 30 mins
2. T0 baseline measurement on Lightcam
3. Product application as per randomization using standard application gesture
4. Subjects to wait in standard condition for 10 mins
5. Timm measurement on Lightcam
6. Subjects to wait in standard conditions for next 4 hrs for subsequent instrumental measurements

The sebum control study results shown in Table 4 indicates that Examples 12 and 13 had significant reduction of sebum, which outperformed some of the best benchmark products on the markets.

TABLE 4

| | Change in gloss shine |
|---|---|
| Ex 13 | −2.47 |
| Ex 12 | −2.48 |

### [Cleansability Study Results]

Cleansability studies were carried out according to the procedure illustrated below. Briefly, first apply the sebollution and take a picture for TO, then dilute the cleanser and apply it on sebollution, and finally massage, take another picture at Ti.

The calculation for cleansability is also illustrated below. The results are shown in Table 5.

As shown in Table 5, Examples 17, 18, 14, and 12 achieved good cleansability, comparable to the cleansability of benchmark cleansing products on the markets.

$$Cleansability = \frac{{De}_{sample cleaned} - {De}_{black reference}}{{De}_{clean reference} - {De}_{black reference}}$$

$${D}_{e} = \sqrt{{D}_{l}^{2} + {D}_{a}^{2} + {D}_{b}^{2}}$$

De: color difference in a rectangular coordinate system
Black reference: bioskin with sebollution applied following pore deep clean protocol
Clean reference: clean bioskin w/o any sebollution

TABLE 5

| | % |
|---|---|
| | Cleansability |
| Ex 17 | 67 |
| Ex 18 | 72 |
| Ex 14 | 88 |
| Ex 12 | 85 |

### [C. Acne Study Results]

The C. acne studies were performed according to the Microbiology department standard procedure I_M_ANA_006_01 “Activity of formulae against microorganisms of cosmetic interest”.

In summary, in a suitable culture medium, we evaluated the biocidal effect on the strain of interest of formulae at 10% after 2 h, 6 h and 24 hours of contact time, in comparison to a control (medium without formula). The reduction in the number of viable bacteria is measured over time. This assay is a “Time Kill” test.

The C. acne results for log reduction compared to the control are shown in Table 6. The C. acne study results for Examples 17, 18, 14, and 12 indicate satisfactory level of reduction.

Note that Examples 17, 18, 14, and 12 had a pH around 5.5. We further observed that the pH of the samples had significant effect on C. acne reduction. The C. acne results for log reduction with effect of pH are shown in Table 7. A lower pH of 5.5 clearly improved the C. acne reduction compared to pH 7.2.

TABLE 6

| | | 2H | 6H | 24H |
|---|---|---|---|---|
| Ex 17 | Acne Paste Cleanser 2% Glycerin | 0.6 | 1 | 2.3 |
| Ex 18 | Acne Paste Cleanser 40% Glycerin | 0.4 | 0.8 | 2 |
| Ex 14 | Acne Paste Cleanser 10% Glycerin | 0.6 | 0.9 | 2 |
| Ex 12 | Acne Paste Cleanser 30% Glycerin | 0.6 | 1 | 2 |

TABLE 7

| | | | Log reduction compared | | |
|---|---|---|---|---|---|
| | | | to the Control | | |
| | | | 2H | 6H | 24H |
| PH | Ex 16 | Acne Paste Cleanser 2% | 1.9 | 3.9 | 5 |
| 5.5 | | Glycerin + 10% Sodium Cocoyl | | | |
| | | Isethionate | | | |
| | Ex 15 | Acne Paste Cleanser 2% Glycerin | 1.5 | 1.4 | 4.1 |
| pH | Ex 16 | Acne Paste Cleanser 2% | 0 | 0.2 | 0.6 |
| 7.2 | | Glycerin + 10% Sodium Cocoyl | | | |
| | | Isethionate | | | |
| | Ex 15 | Acne Paste Cleanser 2% Glycerin | 0 | 0 | 0 |

[Three-In-One Applications of the Acne Paste Formulas]Examples 12, 14 and 18 were developed to be 3-in-1 formulas for a daily skin clearing cleanser when mixed with water, an overnight drying spot treatment in its original form, and a deeply purifying mask when layered on damped face.

Examples 12, 14 and 18 contained optimal concentrations of kaolin, solid mild surfactant (sodium cocoyl isethionate) and glycerin for fast-drying, sebum control, and hydration. They also contained effective amounts of salicylic acid, rhamnolipids, and niacinamide for anti-bacterial and anti-inflammatory treatments; and sufficient amount of chitosan for enhanced salicylic acid deposition.

Moreover, Examples 12, 14 and 18 had a unique wasabi-like paste texture suitable for the 3-in-1 applications, which also require many optimized physical attributes such as spreadability, drying rate, miscibility with water, foaming, rinsibility, and appearance etc. The attributes were rated as acceptable, fair, or good. As shown is Table 8, Examples 12 and 14 had better physical attributes in general than Example 18.

TABLE 8

| | Cleanser | | | | | | Spot Treatment | | |
|---|---|---|---|---|---|---|---|---|---|
| | Miscibility | Quickness | Quickness | Mask | | | | Stay | |
| | w/ | of | of | Spread- | Cover- | | Quick | in | |
| | water | Foaming | Rinsing | ability | age | Rinsibility | Drying | Place | Clear |
| EX | acceptable | acceptable | fair | good | fair | good | acceptable | fair | good |
| #18 | | | | | | | | | |
| EX | good | fair | good | good | fair | good | good | good | fair |
| #14 | | | | | | | | | |
| EX | fair | good | good | good | fair | good | acceptable | fair | good |
| #12 | | | | | | | | | |

1. A cosmetic composition comprising:
(a) at least one glycolipid;
(b) at least one salicylic acid or its derivatives of formula (I): 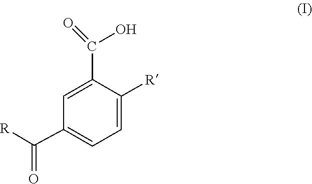
in which:
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
R′ is a hydroxyl group or an ester group of formula: 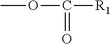
in which R₁ denotes a linear or branched, saturated or unsaturated aliphatic chain containing 1 to 18 carbon atoms; and also salts thereof derived from an inorganic or organic base;
(c) at least one cationic polymer selected from a polysaccharide derived from 3 linked D-glucosamine or N-acetyl-D-glucosamine;
(d) at least one clay;
(e) cosmetically acceptable solvents comprising water, and comprising glycerin present from about 2% to about 40% by weight, relative to the total weight of the composition; and
wherein the pH of the composition is from about 3.5 to about 6.5.

2. The composition according to claim 1, wherein the glycolipid is selected from the group consisting of rhamnolipids, sophorolipids, glucolipids, trehalolipids, cellobiose lipids, and mixtures thereof.

3. The composition according to claim 2, wherein the glycolipid is rhamnolipids.

4. The composition according to claim 1, wherein the amount of the glycolipid(s) is from 0.1% by weight to 10% by weight, relative to the total weight of the composition.

5. The composition according to claim 3, wherein the amount of the rhamnolipids is from 1% by weight to 7% by weight, relative to the total weight of the composition.

6. The composition according to claim 5, wherein the amount of the rhamnolipids is from 2% by weight to 5% by weight, relative to the total weight of the composition.

7. The composition according to claim 1, wherein the salicylic acid or its derivatives is salicylic acid.

8. The composition according to claim 1, wherein the amount of the salicylic acid or its derivatives is from 0.1% by weight to 5% by weight, relative to the total weight of the composition.

9. The composition according to claim 8, wherein the amount of the salicylic acid or its derivatives is from 1% by weight to 3% by weight, relative to the total weight of the composition.

10. The composition according to claim 1, wherein the cationic polymer is chitosan.

11. The composition according to claim 1, wherein the composition is in the form of a paste.

12. A method for treating, hydrating, and/or cleansing acne-prone skin, comprising applying onto the skin in need a composition comprising:
(a) at least one glycolipid; and
(b) at least one salicylic acid or its derivatives of formula (I): 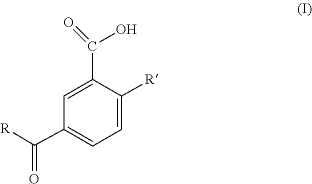
in which:
the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
R′ is a hydroxyl group or an ester group of formula: 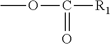
in which R₁ denotes a linear or branched, saturated or unsaturated aliphatic chain containing 1 to 18 carbon atoms; and also salts thereof derived from an inorganic or organic base;
(c) at least one cationic polymer selected from a polysaccharide derived from 3 linked D-glucosamine or N-acetyl-D-glucosamine; and
(d) at least one clay;
(e) cosmetically acceptable solvents comprising water, and comprising glycerin present from about 2% to about 40% by weight, relative to the total weight of the composition; and
wherein the pH of the composition is from about 3.5 to about 6.5.

13. A cosmetic composition comprising:
(a) at least one rhamnolipid present from 2% by weight to 5% by weight, relative to the total weight of the composition;
(b) at least one salicylic acid or its derivatives present from about 1% by weight to about 3% by weight, relative to the total weight of the composition;
(c) at least one cationic polymer comprising chitosan;
(d) at least one clay comprising kaolin;
(e) cosmetically acceptable solvents comprising water, and comprising glycerin present from about 10% to about 30% by weight, relative to the total weight of the composition;
wherein the pH of the composition is from about 3.5 to about 6.5,
wherein the composition is suitable for 3-in-1 paste formulas for acne-prone skin as a skin cleanser, as a spot treatment, and as a mask.

14. The composition of claim 13, further comprising at least one surfactant comprising sodium cocoyl isethionate present at about 10% by weight, relative to the total weight of the composition.

15. The composition of claim 13, further comprising niacinamide present at about 2% by weight, relative to the total weight of the composition.

16. The composition of claim 13, wherein the kaolin is about 15% by weight, based on the weight of the composition.

17. The composition according to claim 13, wherein the composition is a wasabi-like paste.

18. The composition according to claim 13, wherein the composition is a daily clearing skin cleanser to be mixed with water.

19. The composition according to claim 13, wherein the composition is an overnight drying spot treatment in its original form.

20. The cosmetic composition according to claim 13, wherein the composition is a mask to be layered on damped face.
